(19) United States
(12) Patent Application Publication
(10) Pub. No.: US 2024/0198088 A1
(43) Pub. Date: Jun. 20, 2024

(54) ENDOTRACER AND ENDOPACER SYSTEMS AND PROCESSES INCLUDING USING AT LEAST TWO ELECTRODES

(71) Applicant: Pradeep Selvan, Los Angeles, CA (US)

(72) Inventor: Pradeep Selvan, Los Angeles, CA (US)

(21) Appl. No.: 17/803,844

(22) Filed: Dec. 20, 2022

(51) Int. Cl. A61N 1/05; A61B 5/293; A61B 5/37

(52) U.S. Cl. CPC A61N 1/0531; A61B 5/293; A61B 5/37; A61N 1/0534

(57) ABSTRACT

Novel enhanced endovascular tracing and pacing methods and systems using endovascular electrodes are described. Medial Temporal Lobe, SubCortical and Cortical sites within the posterior aspects of the brain are accessed through the Left subclavian and jugular veins into the sinus and venous systems of the brain.

### CROSS-REFERENCE TO RELATED CONTRIBUTIONS

Full Paris Convention & All Other Priority Rights Expressly Claimed from U.S. Provisional Ser. No. 63/474,446 (Aug. 18, 2022) and 63/293,061 (Dec. 22, 2021), by the same applicant/assignee. On knowledge, the present disclosure comprises nothing supported by grant funding, nor any related assistance of any government agency to date.

### BACKGROUND OF THE INVENTIONS

The present inventions relate to improved neurosurgical outcomes through the use of novel endovascular pacing (endopacer) systems, meaning minimally invasively emplaced devices which stimulate and record signals within the brain. In particular, endo pacer direct current stimulation methods are disclosed using two electrodes.

### Objects and Summary of the Inventions

Briefly stated, EndoPacer systems are deployed during neurosurgery to produce better clinical outcomes, and comprise a catheter-system means for access to the endovascular landscape of the brain. Endo Pacer access enables physicians to intervene in various tortuous vessels to enhance patient surgical outcomes.

According to embodiments, there are provided a method of endovascular tracing, constituting progress in science and the useful arts eligible as subject matter for U.S. Letters Patent, inter alia. This process includes at least one electrode in preferred embodiments, and encompasses both known and later developed technology for managing brain signal recording, stimulation and related approaches and methodologies known to artisans upon reading the instant disclosure, as described and claimed below, it is respectfully proposed.

According to embodiments, there is provided a method of endovascular pacing during neurosurgery, interventional procedures and other care provider mediated diagnostic and therapy-delivering interactions, including robotics and hybrid forms of treatment.

According to embodiments, the systems provide the ability to monitor and pace brain signals including with respect to secondary progress of catheters, delivery systems, brain coils, implants, and other medical devices, providing advantages and improved patient outcomes.

According to embodiments, entities such as hospitals, clinics and other healthcare organizations can benefit from this technology.

According to embodiments, EEG signals drive the system and are generated and recorded to improve on users of stereotactic depth electrodes and subdural grid electrodes of the prior art (not shown herein as conventional).

### BRIEF DESCRIPTION OF THE DRAWINGS

Detailed descriptions of preferred embodiments of the invention will be made with reference to the accompanying drawings, in which like reference designators represent common elements, mindful that multiple alternate embodiments of the present invention are both illustrated and contemplated. Likewise, owing to the nature of the local brain environment, structures delineating the same may be labeled in select figures the same being workpieces as defined in the US Patent Laws and not being confused with any claimed elements, it is respectfully submitted.

FIG. 1 is a magnified and schematic cartooned view of an EndoPacer being a pair of endovascular electrodes according to the teachings if the current invention;

FIG. 2 is a magnified schematic cartooned view of the recording and/or impulse generating apparatus onto which the EndoPacer system can be inserted and connected to recording/stimulating mechanism.

FIG. 3 is another magnified schematic and cartooned view of an EndoPacer system being a pair of anode/cathode insertion sites for the leads of the EndoTracer System of the present invention being the connections to power for each said endovascular electrode; and

FIG. 4 is cartooned view of the a lead according to the present invention;

FIG. 5 is a schematic exemplary but not limiting local geographic landscape of the brain;

FIG. 6 likewise shows access and exemplary situses, with detailed excerpts to follow;

FIG. 6A shows emplacement situs at Medial Temporal Lobe;

FIG. 6B shows emplacement situs at Sub Cortex; and

FIG. 6C shows emplacement situs at Cortex;

FIG. 7 further describes brain locations and structures;

FIG. 8 adds detail to the above figures; and does the same to show exemplary but not limiting embodiments schematically cartooned to show what is respectfully proposed to be material innovation.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

### DETAILED DESCRIPTION OF THE INVENTIONS

The present inventor defines for the purpose of this application family and subsequent letters patent the terms “ENDOTRACER” and “ENDOPACER” as proprietary systems combining access and current flow to specifically denoued and otherwise applicable areas of the brain, using at least a described claimed and/or otherwise related electrode set (U.S. Ser. No. 63/293,061) and (U.S. Ser. No. 63/474,446), along with those grammatical innovations within the ambit of those skilled in there art to deliver said signals, along with harvesting, monitoring and all technical steps in between, similarly omitted as conventional, such as power source dimensions and other state of the art teachings which are expressly incorporated herein by reference, meaning that direct current stimulation using endovascular wire electrodes, as shown and described herein and all other identical uses of the same to achieve the same function/way/result are offered for consideration as covered by the terms. Said Inventor has identified unique opportunities to use endovascular tracing and pacing systems during any procedure needing, for example, EEG signal inputs or outputs including numerous approaches known to those skilled in the art. Expressly incorporated are the state of the prior art, namely any and all components and devices from U.S. Pat. Nos. 10,912,93, 10,589,096, 10,458,421, 10,130,940, 9,808,359, 9,420,950, and 8,088,140, along with U.S. Pat. Nos. 11,043,036, and 10,470,741.

The Figures show a schematized schematically simplified and cartooned set of views of an exemplary inventive endovascular electrode devices, which likewise functions as an exemplary device from the provisional patent applications referenced above and the fully described and claimed improved from novel enhanced endotracer/endopacer systems by providing both stimulating and recording capacities.

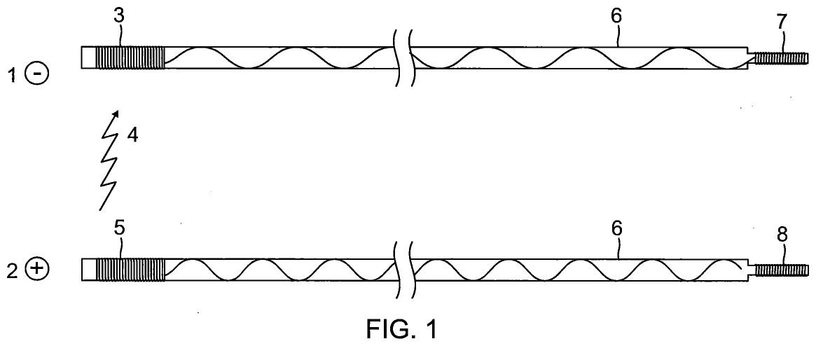

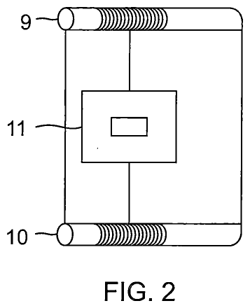

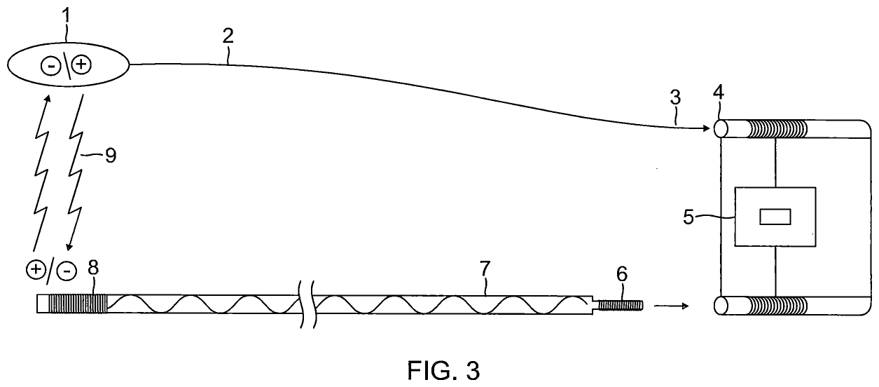

Referring now to FIG. 1, FIG. 2, and FIG. 3, IMPULSE CURRENT GENERATOR 11 interchangeable with signal propagating means of any analogous form, is linked to CATHODE ELECTRODE INSERTION SITE TO GENERATOR 10, and ANODE ELECTRODE INSERTION SITE TO GENERATOR 9, which communicates with CATHODE CONNECTOR ELECTRODE CONNECTOR LEAD 8. Likewise, ANODE ELECTRODE CONNECTOR LEAD 7, links to and is connected with CONNECTOR WIRE 6. ANODE TIP 5, is shown with the DIRECTION OF CURRENT FLOW FROM ANODE TO CATHODE TIP 4, and CATHODE TIP 3, defines the NEGATIVE ELECTRODE (CATHODE) 1, with POSITIVE ELECTRODE (ANODE) 2, likewise being shown.

FIG. 3 shows direct current stimulation using external (scalp or subdural) electrode and endovascular wire electrode. EXTERNAL (SCALP OR SUBDURAL) ELECTRODE (CATHODE OR ANODE) 1, linked by and connected with EXTERNAL ELECTRODE CONNECTOR WIRE 2, EXTERNAL ELECTRODE INSERTION TO GENERATOR/RECORDER 3, along with EXTERNAL ELECTRODE GENERATOR/RECORDER LEAD 4, and ENDOVASCULAR ELECTRODE GENERATOR RECORDER LEAD 5, ENDOVASCULAR ELECTRODE INSERTION TO GENERATOR/RECORDER 6, operationally and electrically connected to ENDOVASCULAR ELECTRODE CONNECTOR WIRE 7, ENDOVASCULAR ELECTRODE TIP (ANODE OR CATHODE) 8, with CURRENT BETWEEN EXTERNAL AND ENDOVASCULAR ELECTRODES 9, completing said circuit.

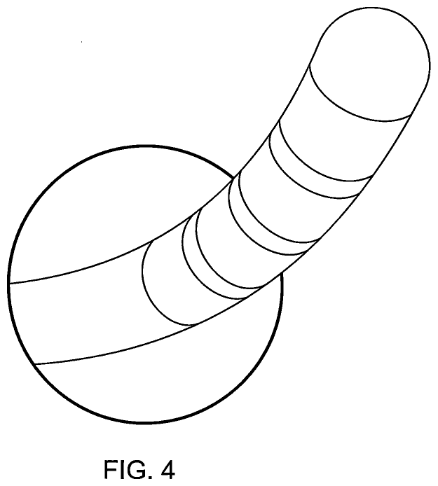

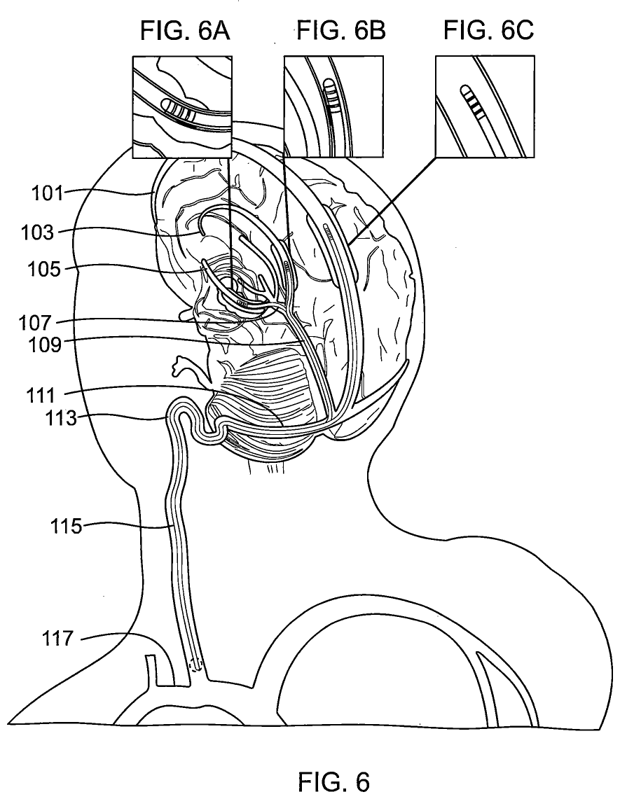

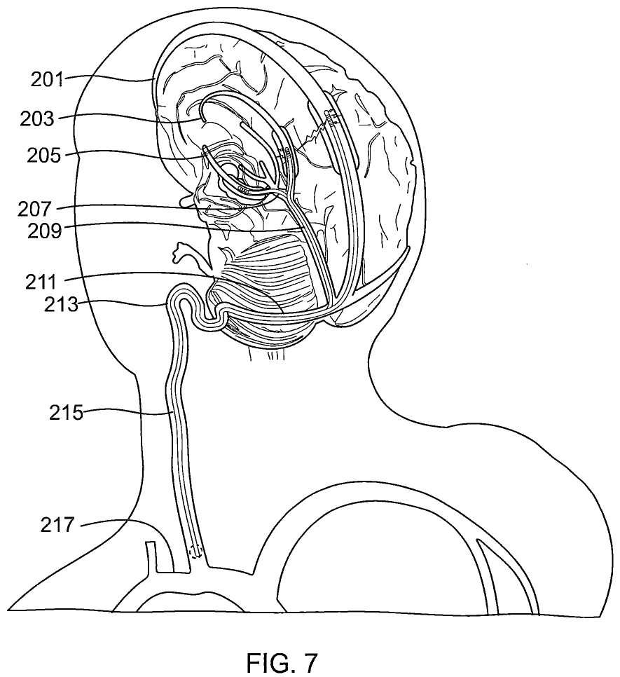

Those skilled in the art understand that signal propagation works for both the endopacing and endotracing applications of the instant technology as further demonstrated and explained with respect to FIGS. 4-8. Likewise, use of single electrode for the latter and direct current stimulation using two endovascular wire electrodes may include scalp-based attachments, other surface elements or implants. While the schematics illustrate the gross surface morphology in a cartooned fashion, Artisans understand that the EndoTracer lead is comprised of for example, four smaller electrodes and used to find and record signals while the Endopacer leads comprise one larger electrode as shown respectively in FIGS. 6 and 7 below.

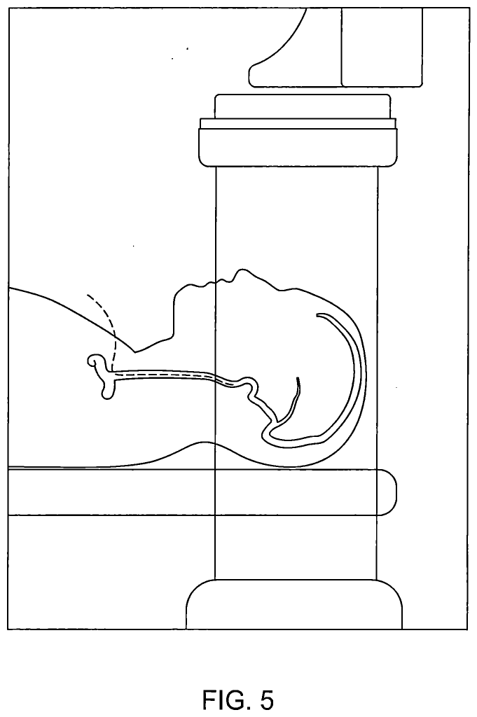

FIGS. 4-8 show the fundamental aspects of each of the disclosed, explained and claimed systems, with anatomical signposts left in have text labels for ease of understanding. FIG. 4 indicates distal end of lead wire, while FIG. 5 shows a patients' posture on as table in the operations theatre with imaging (fluoroscopy, etc. . . . ) being focused on the subject brain areas being addressed.

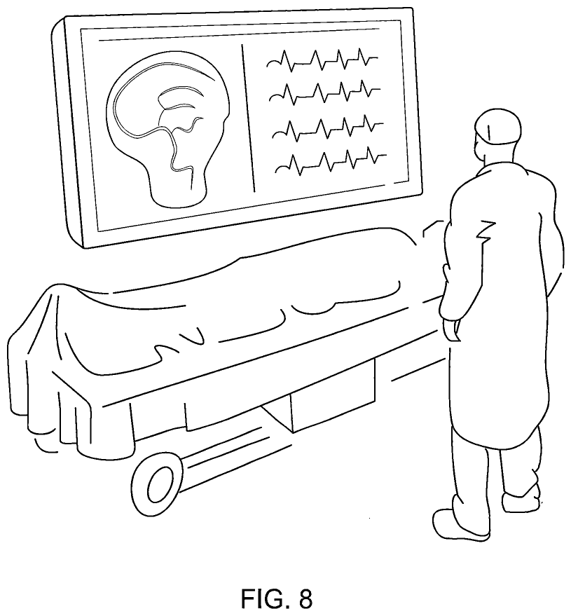

Referring now to FIG. 6, schematic and cartooned versions of a reverse, lateral and partial perspective view is shown of both procedures, with endotracing first featured for ease of discussion. Sites for respective emplacement comprise MEDIAL TEMPORAL LOBE 6A, SUB CORTEX 6B and CORTEX 6C in exploded and detailed views show the path of travel of the instant system which is accessed via the Left subclavian vein 117, providing ingress into the Internal jugular vein 115. Traveling up the neck to the base of the brain the Sigmoid Sinus 113, leads to the Transverse Sinus 111, and orthogonal to the same the Straight Sinus 109 is followed to both the Basal vein 105 (through the Vein of Galen 107) and the SUBCORTEX (6B), traveling distal Part way down the Inferior sagittal sinus. Likewise, traveling toward the Inferior sagittal sinus 103 CORTEX (6C) placement is used. The endotracer system find and records signals form the brain and transmits them externally (see FIG. 8).

Referring now to FIG. 7 and to FIG. 8, EndoPacing is a process by which an identical access path for example, as discussed in FIG. 6 above is followed, whereby direct current stimulation using endovascular wire electrodes (as shown in FIG. 1, 2) is initiated. The processes as described and claimed here includes access through the left subclavian vein 217, upwards through the neck to the Internal jugular vein 215, followed by a corner bend into the Sigmoid sinus 213, all of the way to the midpoint of the transverse sinus 211, wherein with an orthogonal rotation the Straight sinus 209, yields all three previously disclosed targets via the Vein of Galen 207 to the Basal vein 205, and into the Inferior sagittal sinus 203, where the signals are propagated and transmitted to device emplaced within the Superior sagittal sinus 201 as shown between the respective electrodes in the figures.

FIG. 8 shows that the present inventions both generate and record EEG-type of signals to show local and regional states of brain activity. By using trans-venous access to the jugular vein, the internal cerebral veins and sinuses can be used for recordation and stimulation.

Likewise, those skilled in the art recognize that placement of novel enhanced endotracer and endopacer systems each is achieved via the techniques of U.S. Pat. No. 10,398,877, or any combination of techniques, as illustrated herein, along with known trans-femoral and radial access, for example from U.S. Pat. No. 8,070,791 8,088,140; 8,197,743; 8,545,514; 8,585,713, as if funnily set further herein.

Logical combinations and subcombinations of one and two electrode systems with external and internal placement of Generators is contemplated as not disclosed before to one knowledge in the art.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using, consisting of, or consisting essentially of language. When used in the claims, whether as filed or added per amendment, the transition term “consisting of” excludes any element, step, or ingredient not specified in the claims. The transition term “consisting of” essentially limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for direct current stimulation using endovascular electrodes, which comprises, in combination:
an electrode or at least two electrodes being anodes and cathodes having distal tips for detecting, recording and transmitting electrical signals;
connector wires; and
an impulse current generator;
wherein the components are all electrically connected and current flows from anode to cathode tip, within the brain at predetermined locations.

2. The system of claim 1, wherein the impulse current generator is at least one of implanted, placed on or within the scalp, and otherwise within electrically connectable proximity to an electrode pair, set or single electrode.

3. The system of claim 2, for performing at least one procedure form the group consisting of finding, recording or transmitting signals (endotracing) and generating signals to be transmitted within a brain (endopacing).

4. The system of claim 3, wherein concurrent imagery is being used to confirm data relative to the positioning of each said electrodes.

5. The system of claim 4, wherein access to the cerebral sinus and venous system is through the Left subclavian vein, the Internal Jugular vein to the sigmoid sinus.

6. The system of claim 5, further comprising using the transverse sinus to access at of the vein of Galen and and Basal vein to access the Medial Temporal Lobe.

7. The system of claim 6, further comprising access to the SubCortex via the Inferior sagittal sinus.

8. The system of claim 7, further comprising access to the Cortex via the Superior sagittal sinus.

9. A process for endovascularly implanting at least one of an electrode and an electrode set with the brain, which comprises, in combination:
providing an access tool set for creating an ingress and entering the Left subclavian vein with a lead wire;
traveling distally from the access site into the Internal jugular vein in the direction of the Sigmoid sinus;
traversing the Sigmoid sinus through to its midpoint;
proceeding orthogonally from the rear of the brain toward the midbrain via the Straight sinus;
passing through the Vein of Galen and emplacement within the Basal vein of the Medial Temporal Lobe of the brain.

10. The process of claim 9, wherein the step of passing by-passes the Vein of Galen, and emplacement happens in the SubCortex of the brain, part way toward the terminus of the Inferior Sagittal sinus.

11. The process of claim 10, wherein the the electrode is emplaced within the Cortex of the brain traveling distally within the Superior sagittal sinus.

12. The process of claim 11, wherein the at least an electrode finds, records and transmits EEG/electrical signals to an outside source to be displayed and provide data to a care-giver, or endotracing.

13. The process of claim 12, wherein the at least an electrode set generates EEG/electrical signals in conjunction with an impulse current generator in communication therewith.

14. An endovascular electrode, for endotracing comprising, in combination:
at least a neurovascular recording lead effective for endovascular insertion/introduction proximate to the brain;
said at least a neurovascular recording lead further comprising an introduction shaft;
a circumferential electrode;
a wire-electrode connector;
a wire within the introduction shaft;
a surface wire;
a braided wire; and
a proximal wire to an external recording device.

15. Endovascular wire electrodes for delivering direct current stimulation to the brain for endopacing, further comprising:
a negative electrode (cathode);
a positive electrode (anode);
a cathode tip;
an anode tip;
a connector wire;
an anode electrode connector lead; and
a cathode electrode connector lead.

16. The endovascular wire electrodes of claim 15, further comprising:
ready connectability to an anode insertion site to an Impulse current generator; ready connectability to a cathode insertion site to an Impulse current generator; and
An Impulse current generator.

17. The endovascular wire electrodes of claim 16, wherein the Impulse current generator may be implanted, emplaced or positioned within the scalp.

18. The System of claim 8, targeting a plurality of sites between the endovascular insertion/introduction site in the left Subclavian vein and disposed about the hippocampus, which further comprises the Medial Temporal Lobe, the SubCortex and Cortex of the brain.

19. The system of claim 18, which detects, records and transmits electrical signals manifested in tangible media of expression as EEG signals to be interpreted for diagnosis, therapy and deviations from standard singnals to treat patients.

20. The system of claim 19, further comprising:
processing, manipulating and extrapolating from patient blind and HIPAA compliant data sets to create databases and process said data with human-guided AI, optionally, to optimize health care endpoints and objectives
